(19) United States
(12) Patent Application Publication
(10) Pub. No.: US 2012/0109147 A1
(43) Pub. Date: May 3, 2012

(54) Uterine Manipulators and Related Components and Methods

(75) Inventors: Robert D. Auerbach, Madison, CT (US); Charles Sherts, Westport, CT (US); Peter K. Arneson, Cheshire, CT (US); Kerry Blair, Overland Park, KS (US)

(73) Assignee: COOPERSURGICAL, INC., Trumbull, CT (US)

(21) Appl. No.: 13/230,492

(22) Filed: Sep. 12, 2011

Related U.S. Application Data
(60) Provisional application No. 61/408,814, filed on Nov. 1, 2010.

(51) Int. Cl. A61B 17/42

(52) U.S. Cl. 606/119

(57) ABSTRACT

A colpotomizer cup includes a cup body and one or more tenaculum engagement features. The cup body is configured to receive a cervix. The one or more tenaculum engagement features are adapted to connect the colpotomizer cup to a tenaculum such that the colpotomizer cup can be delivered to a cervix using the tenaculum. The colpotomizer cup can be configured to accept and secure the base of a uterine manipulator tip.

### CROSS-REFERENCE TO RELATED APPLICATIONS

This application claims the benefit of U.S. Provisional Application No. 61/408,814, filed on Nov. 1, 2010, which is incorporated by reference.

### TECHNICAL FIELD

This disclosure relates to uterine manipulators and related components and methods.

### BACKGROUND

Uterine manipulators are medical instruments that are used for manipulating (e.g., moving or repositioning) a patient's uterus during medical procedures. Such procedures include surgical procedures such as laparoscopic gynecologic surgery, e.g., total laparoscopic hysterectomy (TLH) surgery.

Instruments of this kind often include a proximal portion that remains external to the patient's body during use and a distal portion that is inserted into the patient's body. The proximal portion typically provides for manipulation of the instrument during use. The distal portion often includes a tip that is sized to be inserted into and/or engage a uterus. Generally, the distal portion of the instrument is advanced through the vaginal cavity and into the uterus. With the distal portion inserted within a uterus, the uterus can be manipulated through surgeon or physician controlled movements of the proximal portion. Following completion of a procedure, the instrument is removed from the patient's body via the vaginal cavity.

### SUMMARY

In general, this disclosure relates to uterine manipulators and related components (e.g., colpotomizer cups) and methods. The uterine manipulators can be used, for example, for manipulating a patient's uterus during gynecological surgery and/or gynecological diagnostic procedures.

In one aspect, a colpotomizer cup includes a cup body configured to receive a cervix, and one or more tenaculum engagement features adapted to connect the colpotomizer cup to a tenaculum such that the colpotomizer cup can be delivered to a cervix using the tenaculum.

In another aspect, a colpotomizer cup delivery assembly includes a tenaculum and a colpotomizer cup. The colopotmizer cup is connected to the tenaculum such that the colpotomizer cup can be delivered to a cervix using the tenaculum.

In a further aspect, a method includes inserting a colpotomizer cup into a vaginal cavity such that the colpotomizer cup receives a cervix; and then inserting a manipulator tip of a uterine manipulator into the vaginal cavity such that a finger of the manipulator tip extends into an opening of the cervix. 17. The method of claim 16, further comprising clamping the colpotomizer cup to the manipulator tip.

Implementations may include one or more of the following features.

In some implementations, the one or more tenaculum engagement features are adapted to releasably connect the colpotomizer cup to a tenaculum such that the colpotomizer cup can be delivered to a cervix using the tenaculum.

In certain implementations, the colpotomizer cup also includes one or more anchors. The one or more anchors are operable to secure the colpotomizer cup to a cervix.

In some implementations, the colpotomizer cup includes a clamping mechanism. The clamping mechanism is operable to secure the colpotomizer cup to a manipulator tip of a uterine manipulator.

In certain implementations, the clamping mechanism includes tenaculum engagement features for connecting the colpotomizer cup to the tenaculum.

In some implementations, the clamping mechanism includes a spring clamp.

In certain implementations, the colpotomizer cup includes one or more tenaculum engagement features that are adapted to releasably connect the colpotomizer cup to the tenaculum such that the colpotomizer cup can be delivered to a cervix using the tenaculum.

In some implementations, the tenaculum is operable to control operation of the one or more anchors.

In certain implementations, the tenaculum includes anchors. The anchors are operable to secure the tenaculum and the connected colpotomizer cup to a cervix.

In some implementations, the tenaculum is operable to control operation of the clamping mechanism.

Methods can also include clamping the colpotomizer cup to the manipulator tip.

In some implementations, the tenaculum is maneuvered to clamp the colpotomizer cup to the manipulator tip.

In certain implementations, the tenaculum is maneuvered to operate a clamping mechanism and thereby clamping the colpotomizer cup to the manipulator tip.

Methods can also include maneuvering the tenaculum to secure the colpotomizer cup to the cervix.

In some cases the tenaculum is maneuvered to engage the cervix with one or more anchors.

Methods can include removing the tenaculum from the vaginal cavity while the manipulator tip and the colpotomizer cup remain in contact with the cervix.

In some cases, inserting the colpotomizer cup into the vaginal cavity comprises inserting a tenaculum carrying the colpotomizer cup into the vaginal cavity and such that the colpotomizer cup receives the cervix.

Implementations may provide one or more of the following advantages.

The placement of a colpotomizer cup about a cervix, prior to insertion of a uterine manipulator tip, can allow for unobstructed placement of the colpotomizer cup. A surgeon can simply, with no obstructions, position the colpotomizer cup on the cervix.

Inserting a colpotomizer cup using a tenaculum allows the colpotomizer cup to be tilted, which can allow for easier insertion.

In some case, loading a colpotomizer cup first, i.e., before placement of a uterine manipulator tip, can help to make the positioning of the colpotomizer cup onto the cervix faster and can also provide for faster positioning of the cervical os, via manipulation of the tenaculum, to allow faster insertion of a uterine manipulator tip into the cervix.

With a simple and single piece design with clearly apparent loading features, a colpotomizer cup can be provided that is quickly and easily loaded onto a tenaculum with low likelihood of misloading error, and thus, can require only minimal training.

A disposable one-piece plastic molded colpotomizer cup can be inexpensively manufactured and a reusable tenaculum can help to minimize medical waste.

Other aspects, features, and advantages are in the description, drawings, and claims.

### DESCRIPTION OF DRAWINGS

FIGS. 1A and 1B are back and front perspective views, respectively, of a colpotomizer cup delivery assembly.

FIG. 2A is a top view of a colpotomizer cup of the colpotomizer cup delivery assembly of FIG. 1.

FIG. 2B is a side view of the colpotomizer cup of FIG. 2A.

FIG. 2C is an end view of the colpotomizer cup of FIG. 2A.

FIGS. 3A and 3B are back and front perspective views, respectively, of a tenaculum of the colpotomizer cup delivery assembly of FIG. 1.

FIG. 4 is a side view of a manipulator handle with a manipulator tip mounted thereon.

FIG. 5 is a side view of a uterine manipulator.

FIG. 6 is an anterior, cross-sectional view of a uterus showing a fully inserted colpotomizer cup delivery assembly.

FIG. 7 is a cross-sectional side view of a pelvic cavity showing the fully inserted colpotomizer cup delivery assembly.

FIG. 8 is an anterior, cross-sectional view of a uterus showing a fully inserted uterine manipulator.

FIG. 9 is a cross-sectional side view of a pelvic cavity showing the fully inserted uterine manipulator.

FIG. 10A is a top view of a colpotomizer cup with integral anchors.

FIG. 10B is a side view of the colpotomizer cup of FIG. 10A, taken along line 10B-10B.

FIG. 10C is a back end view of the colpotomizer cup of FIG. 10A.

FIG. 10D is a front end view of the colpotomizer cup of FIG. 10A.

FIG. 11 is tenaculum, which can be utilized with the colpotomizer cup of FIG. 10A as a colpotomizer cup delivery assembly.

FIGS. 12A and 12B are back and front perspective views, respectively, of a colpotomizer cup delivery assembly including the colpotomizer cup of FIG. 10A and the tenaculum of FIG. 11.

### DETAILED DESCRIPTION

A colpotomizer cup delivery assembly can be used for female pelvic surgical procedures, e.g., during a laparoscopic hysterectomy (TLH) surgery, for positioning a colpotomizer cup about a cervix prior to insertion of a manipulator tip and supporting shaft of a uterine manipulator. This “cup first” method can allow for reduced obstructions when placing the colpotomizer cup about the cervix. Means are also provided for maneuvering the colpotomizer cup, after it is positioned about the cervix, which can allow for relatively easy insertion of the manipulator tip into the cervix.

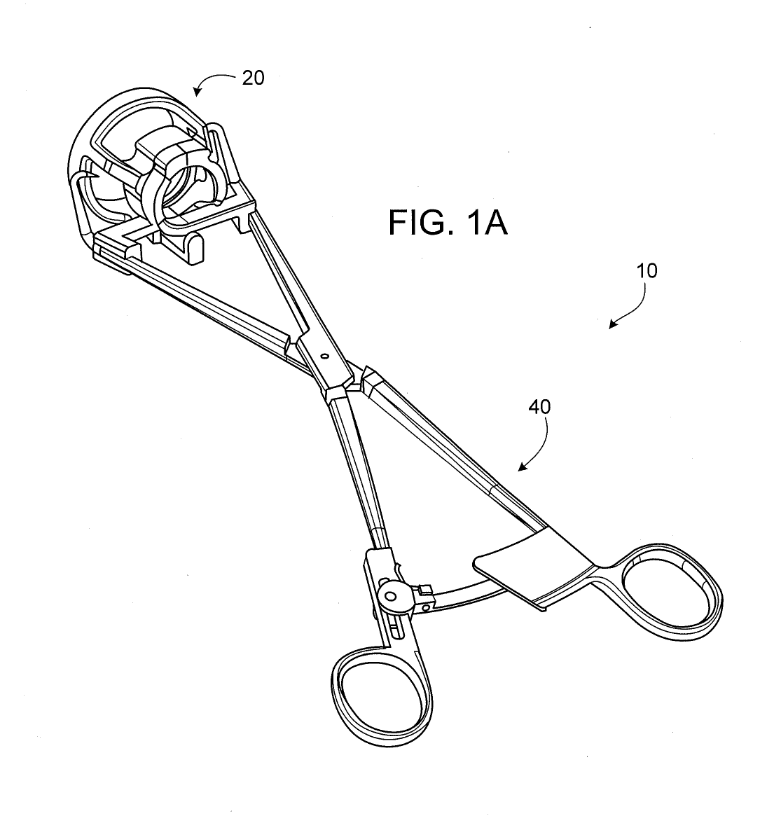

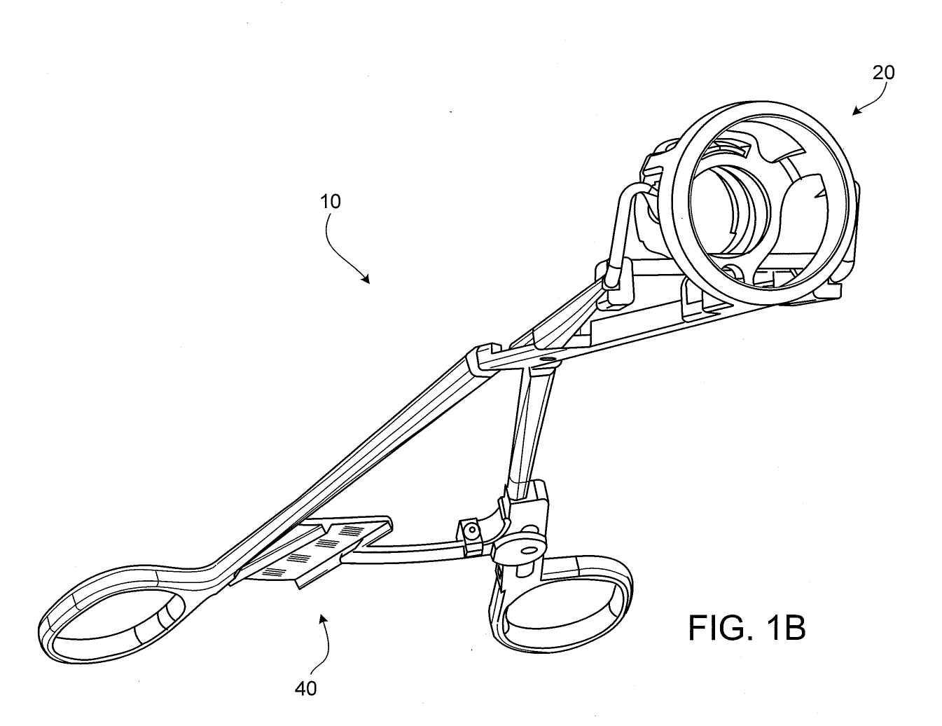

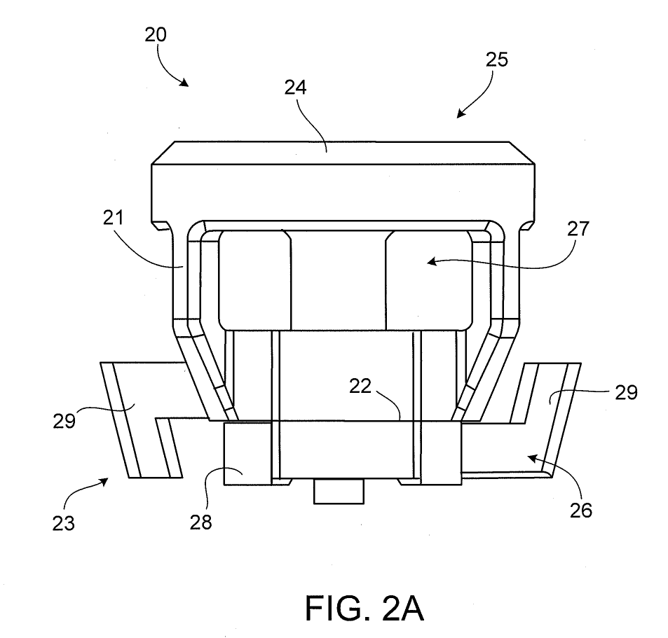

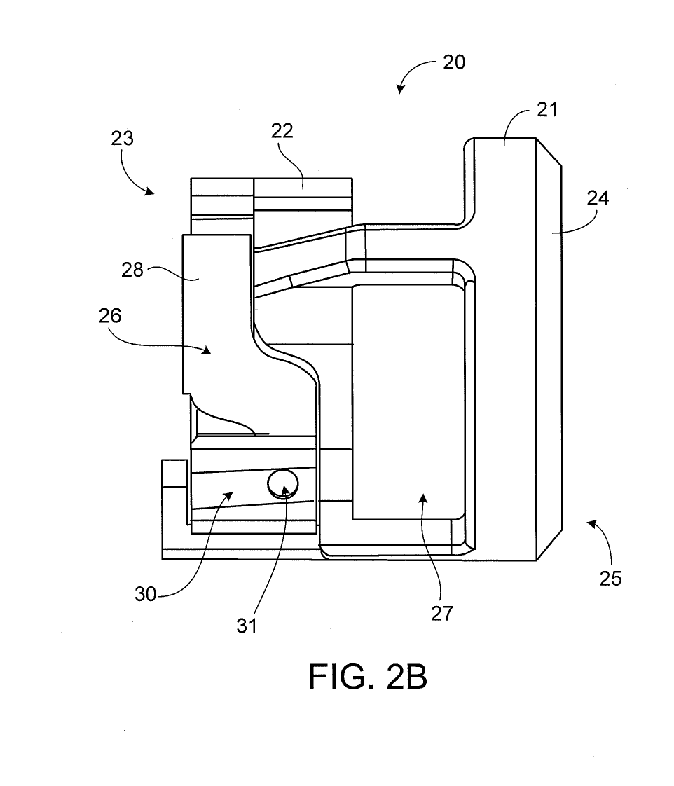

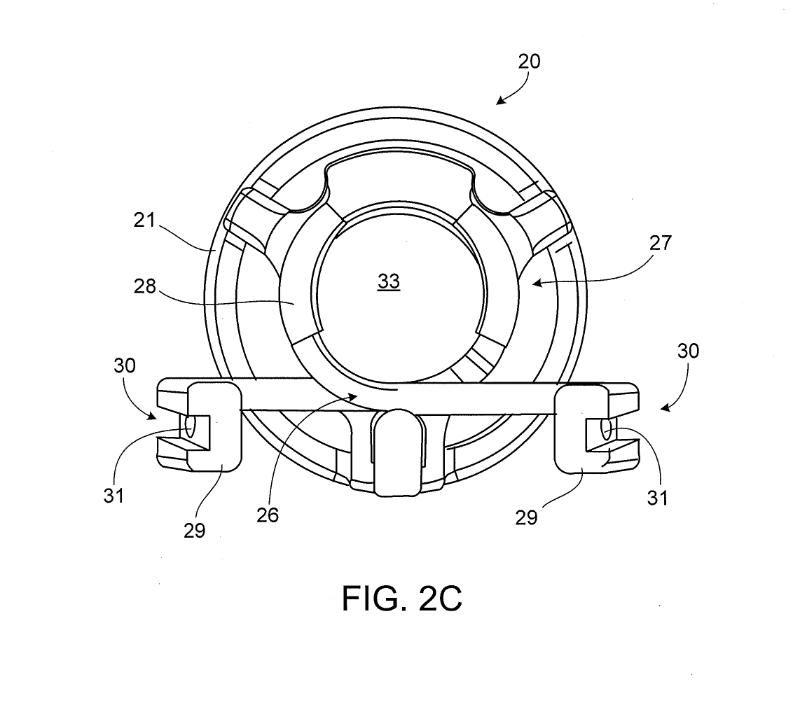

FIGS. 1A and 1B illustrate a colpotomizer cup delivery assembly 10, which includes a colpotomizer cup 20 and a tenaculum 40. Referring to FIGS. 2A-2C, the colpotomizer cup 20 includes an annular body 21 (“cup body”), a cup base 22 at a proximal end 23, and a rim 24 at a distal end 25. A clamping mechanism 26 is connected to cup base 22. The distal end 25 of the colpotomizer cup 20 is configured to receive a cervix, and the rim 24 is beveled to provide an anatomical landmark and incision backstop during use. Viewing windows 27 extend through the annular body 21 and allow for additional visual confirmation of placement of the colpotomizer cup 20 during use.

At the proximal end 23, the clamping mechanism 26 allows the colpotomizer cup 20 to be secured to a manipulator tip of a uterine manipulator. The clamping mechanism 26 includes a spring clamp 28 and a pair tenaculum engagement members 29 that are arranged at opposite ends of the spring clamp 28. The engagement members 29 allow the colpotomizer cup 20 to be loaded on the tenaculum 40, which can then be used to maneuver the colpotomizer cup 20 into position about a cervix.

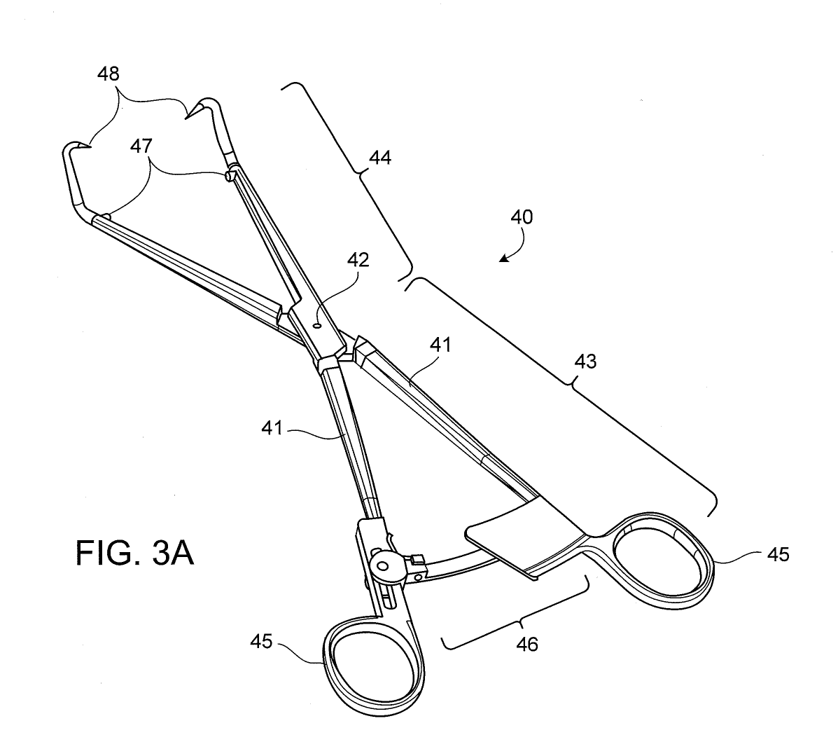

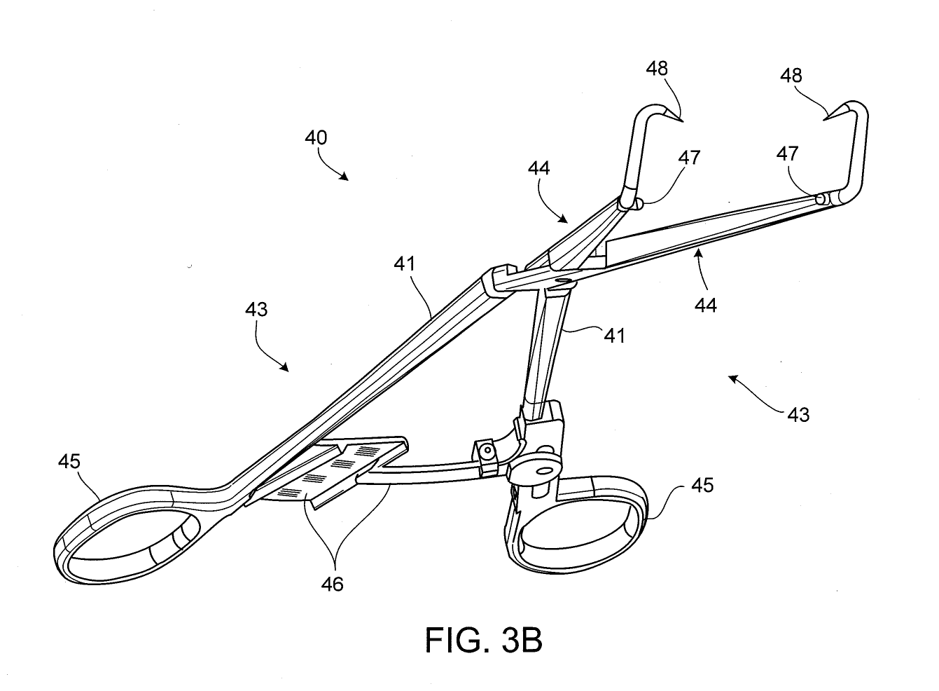

Referring to FIG. 2C, the tenaculum engagement members 29 include recesses 30, which are sized and shaped to receive distal leg portions 44 of the tenaculum, and apertures 31, which are adapted to receive pins 47 (FIGS. 3A & 3B) on inner surfaces of the distal leg portions 44 and which help to inhibit lateral movement of the tenaculum 40 relative to the colpotomizer cup 20.

The annular body 21 has an internal diameter of about 2 cm to about 4 cm (e.g., 2.0 cm, 2.5 cm, 3.0 cm, 3.5 cm, 4.0 cm, etc.). The colpotomizer cup 20 is formed a material suitable for medical devices, that is, medical grade material. Plastics, such as polyvinylchloride, polycarbonate, polyolefins, polypropylene, polyethylene or other suitable medical grade plastic, or metals, such as stainless steel or aluminum can be used. The clamping mechanism 26 can be molded in place on the annular body 21.

As shown in FIGS. 3A and 3B, the tenaculum 40 has a pair of legs 41 that are connected at a common pivot point 42 in a scissor-like construction. Each of the legs 41 includes a proximal leg portion 43 and a distal leg portion 44. The proximal end portions 43 each include a finger ring 45, which allows a surgeon to manipulate the tenaculum 40, and a respective half of a ratcheting mechanism 46. The ratcheting mechanism 46 allows the legs 41 to be locked, relative to each other, in one or more different positions. The distal leg portions 44 each include an associated one of the pins 47, which, as mentioned above, engage the apertures 31 of the tenaculum engagement members 29 to help inhibit lateral movement of the tenaculum 40 relative to the colpotomizer cup 20. The distal leg portions 44 also include anchors 48 which are used to hold the colpotomizer cup 40 in position on a cervix and which also allow the tenaculum 40 to be used to position the cervical os to allow for easy insertion of a manipulator tip into a cervix.

The legs 41 of the tenaculum 40 can be formed, e.g., molded or machined, from materials that are biocompatible and capable of withstanding medical device sterilization procedures, such as heat-based methods (e.g., autoclave, steam autoclave, or dry heat oven) so that the tenaculum 40 as a whole is reusable. Suitable materials that are capable of withstanding medical device sterilization procedures include metals, such as stainless steel and aluminum, and polymers, such as polyoxymethylene (POM) commonly known under the DuPont™ brand name Delrin®.

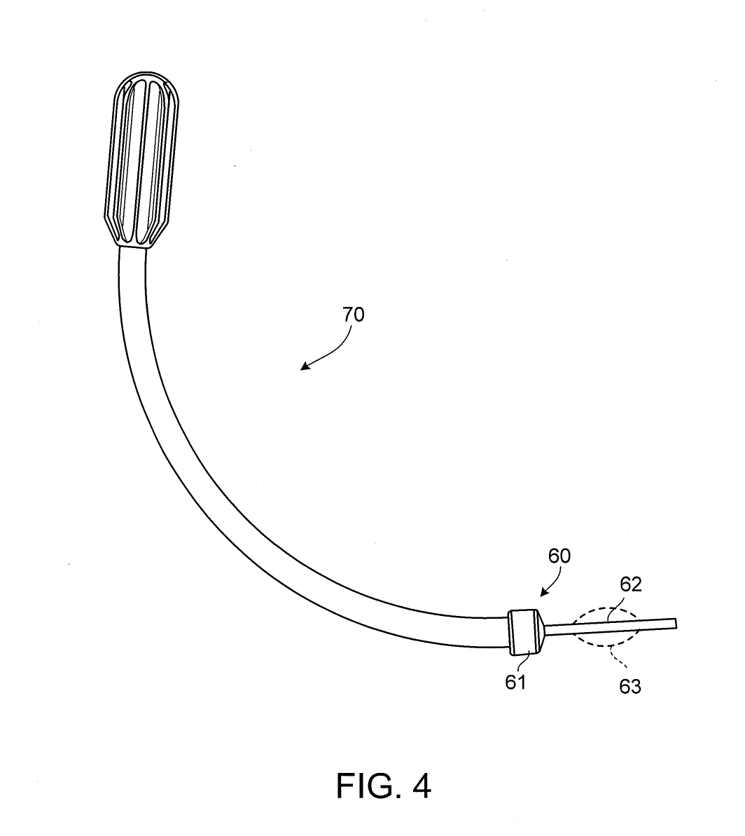

FIG. 4 illustrates a manipulator tip 60 that is releasably coupled to a manipulator handle 70. Suitable manipulator tips are commercially available from CooperSurgical, Trumball, Conn., under the RUMI® tips mark, such as CooperSurgical item numbers UMW676, UMB678, UMG670, and UML516. Suitable manipulator handles are commercially available from CooperSurgical, Trumball, Conn., under the names RUMI® handle and RUMI Arch™.

The manipulator tip 60 includes a tip base 61 and a finger 62 that extends from a first surface of the tip base 61. The finger 62 is configured (e.g., sized and shaped) for insertion into a cervix. The finger 62 carries an expandable balloon 63 for engaging a cervix and/or uterus following insertion. A catheter tube 64, extending from the tip base 61, is in fluid communication with the expandable balloon 63, for inflating the expandable balloon 63.

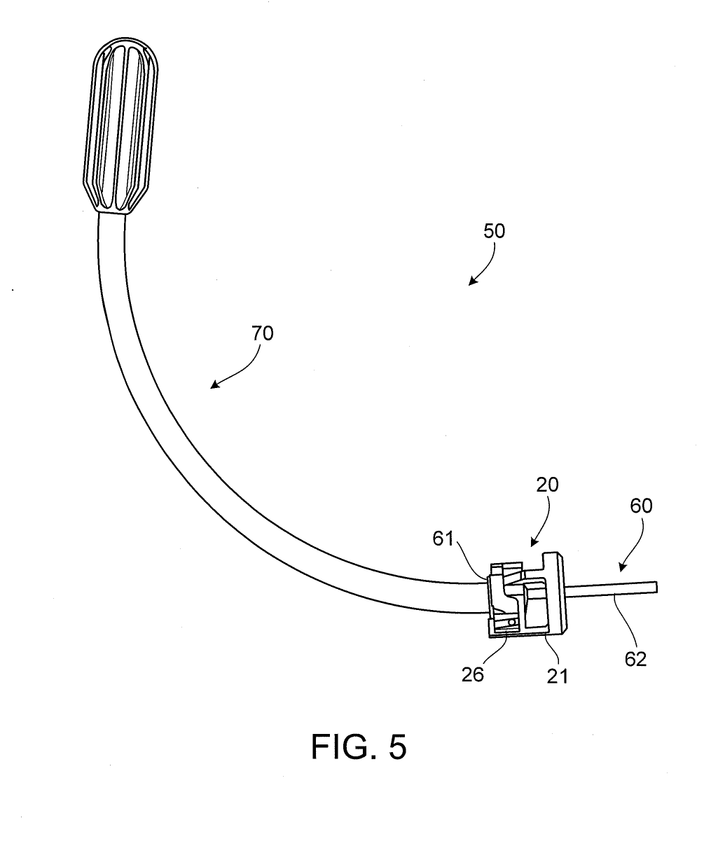

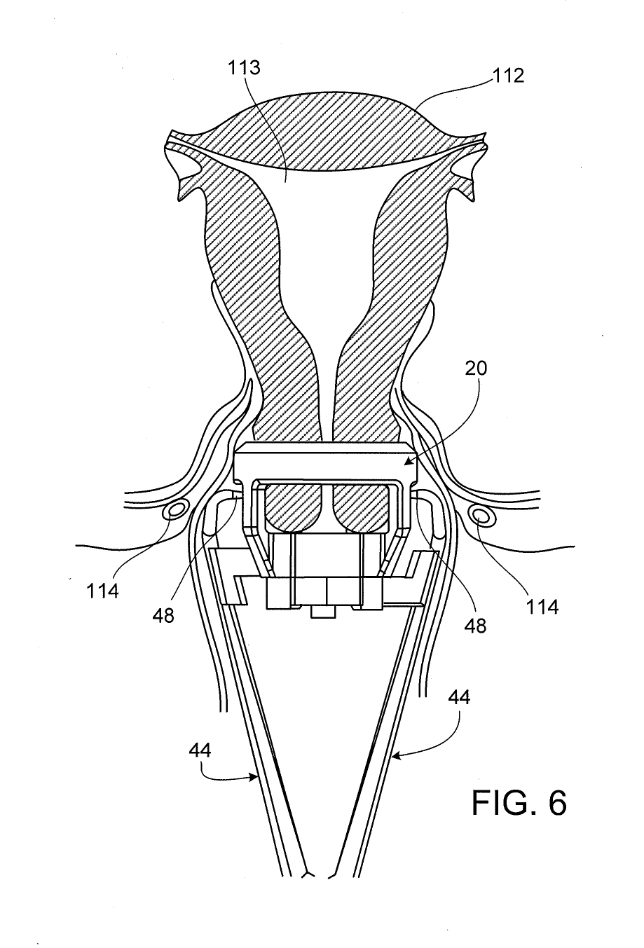

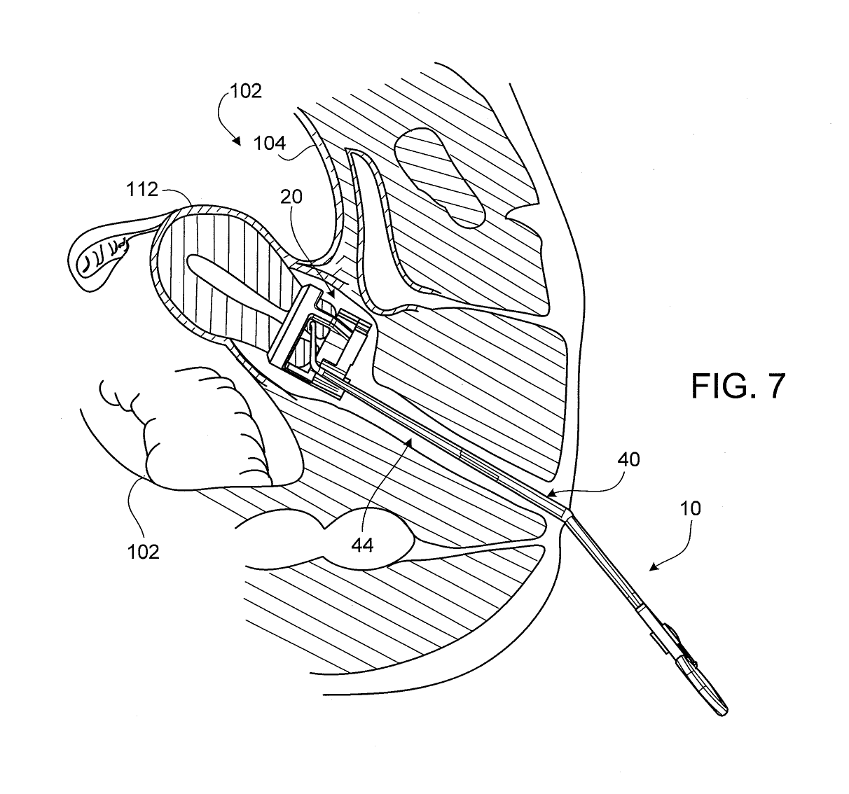

FIG. 5 illustrates a uterine manipulator 50 that includes the colpotomizer cup 20, the manipulator handle 70, and the manipulator tip 60. The colpotomizer cup 20 is mounted to the manipulator tip 60 such that the clamping mechanism 26 engages the tip base 61 and the finger 62 extending through the annular body 21. The colpotomizer cup 20 can be mounted to the manipulator tip 60 by squeezing the ends of the spring clap 28 towards each other, via operation of the tenaculum 40, to enlarge an opening 33 (FIG. 2C) formed by the spring clamp 28. Then, with the opening 33 enlarged, the manipulator tip 60 is inserted, finger 62 first, through the opening 33 and into a position in which the spring clamp 28 and the cup base 22 concentrically surround the tip base 61. With the tip base 61 positioned within the opening 33, the ends of the spring clamp 28 are released allowing the opening 33 to close to a relaxed position in clamping engagement with the tip base 61.

### Methods of Use

The colpotomizer cup 20 may be used in a number of procedures that require manipulation of the uterus, including surgical procedures, such as hysterectomies. In one example, the colpotomizer cup 20 is used in a total laparoscopic hysterectomy (TLH) surgery. A patient is prepared for TLH surgery according to know procedures. Once prepared, the colpotomizer cup 20 is inserted into the vaginal cavity 106 and is advanced towards the cervix 108 using the tenaculum 40. In a fully advanced position, the cervix 108 is received into the annular body 21 of the colpotomizer cup 20 and the rim 24 is place into engaging relationship with the apex 109 of the fornix 110. In this position, the colpotomizer cup 20 provides an anatomical landmark at the base of the uterus 112 (i.e., where the cut is to be made) and also helps to inhibit unintended damage to the ureters 114 by pushing them out of the way. Once the colpotomizer cup 20 is properly positioned, the finger rings 45 of the tenaculum 40 are squeezed towards each other causing the distal leg portions 44 of the tenaculum 40 to close and causing the anchors 48 to pierce the cervix 108 and, at the same time, enlarging the opening 33 of the spring clamp 28. The anchors hold the colpotomizer cup 20 in position relative to the cervix 108. The tenaculum 40 can then be easily manipulated to position the cervical os and allow easy insertion of the manipulator tip 60.

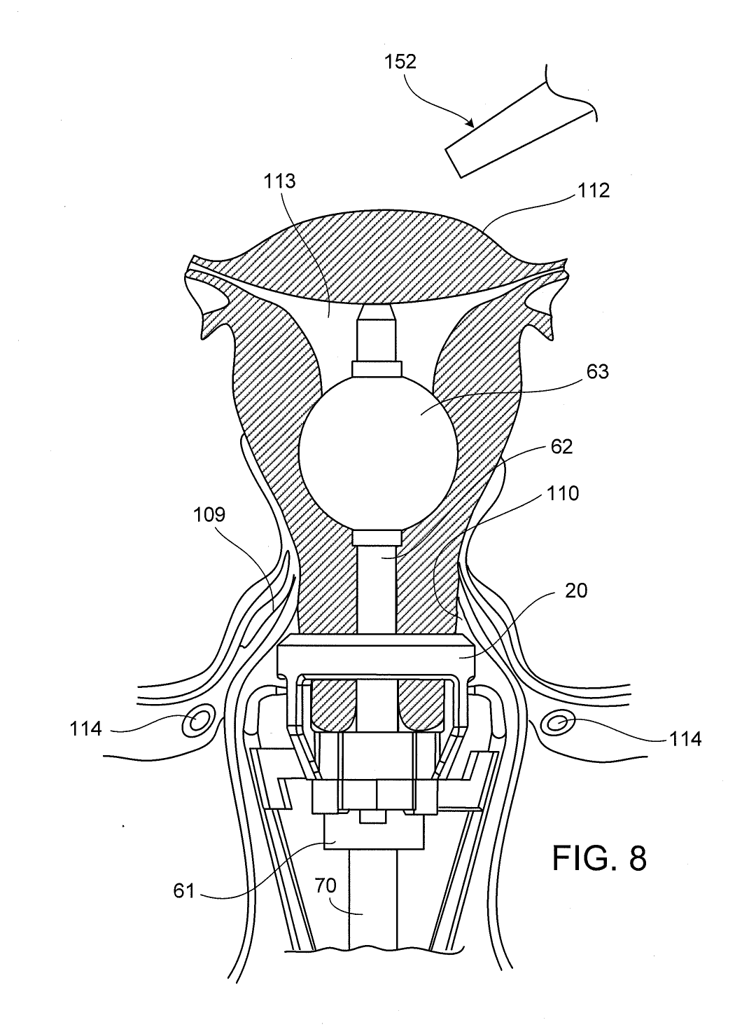

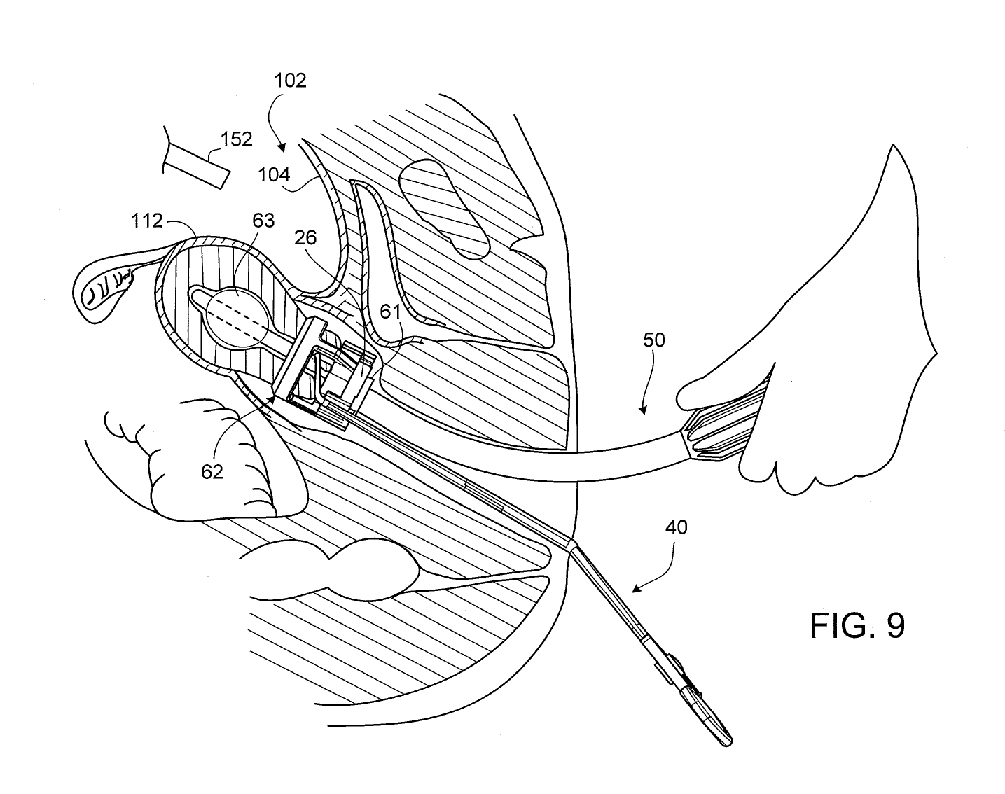

Next, referring to FIGS. 8 and 9, the manipulator tip 60 is inserted into the vaginal cavity 106 using the manipulator handle 70. When inserted, the finger 62 extends into the cervical canal 117 and the tip base 61 is positioned within the opening 33 of the spring clamp 28.

Referring to FIG. 9, the expandable balloon 63 on the finger 62 is inflated (e.g., with sterile, water-based liquid or gas) to hold the manipulator tip 60 and colpotomizer cup 20 in place relative to the cervix 108. The tenaculum 40 is then released causing the spring clamp 28 to lock onto the tip base 61 and the tenaculum 40 is removed from the vaginal cavity 106.

Next, the patient's peritoneal cavity 102 is inflated with a gas (e.g., CO2) to facilitate accessibility and visibility of the female pelvic organs, and surgical instruments, including a laparoscope 152, are inserted through the abdominal wall 104 into the peritoneal cavity 102, as shown in FIGS. 8 and 9. A surgeon can then manipulate or move the uterus 112 into a desired position to perform surgery to cut around the base of the uterus. After the uterus 112 is completely incised such that the uterus 112 is totally free in the peritoneal cavity 102 and held only by the uterine manipulator 50, then the uterine manipulator 50, and the uterus 112, is removed through the vagina.

### Other Implementations

While certain implementations have been described above, other implementations are possible.

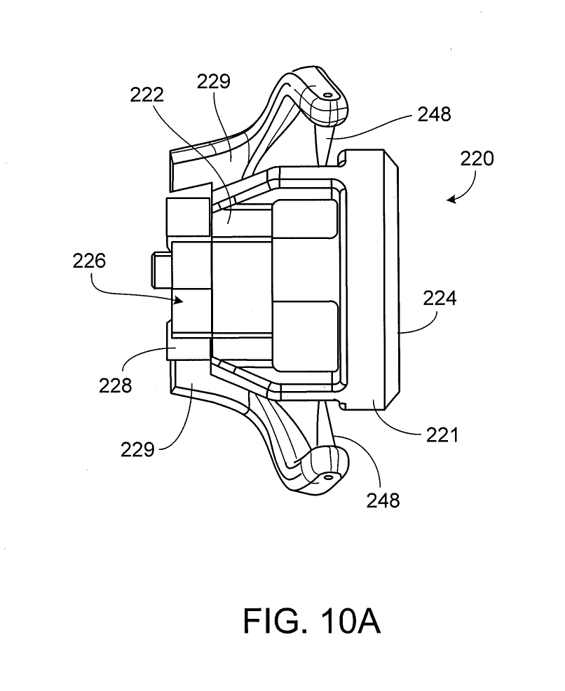

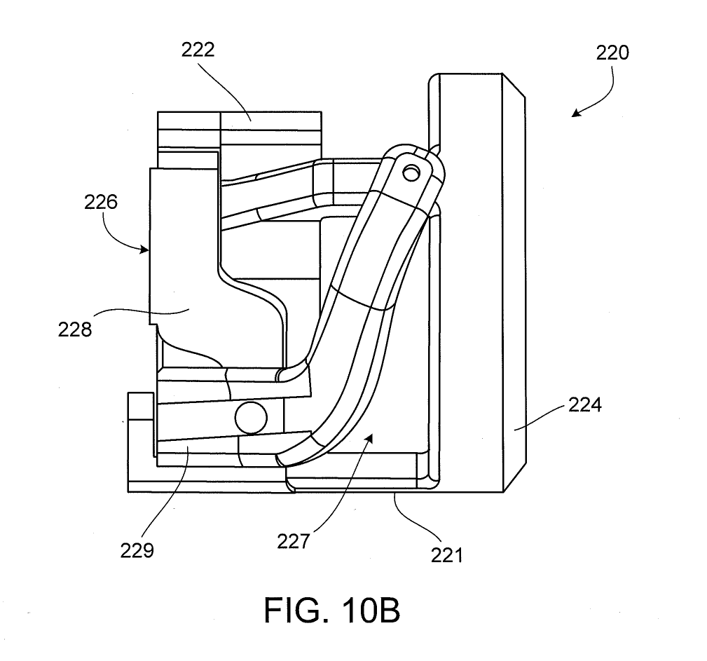

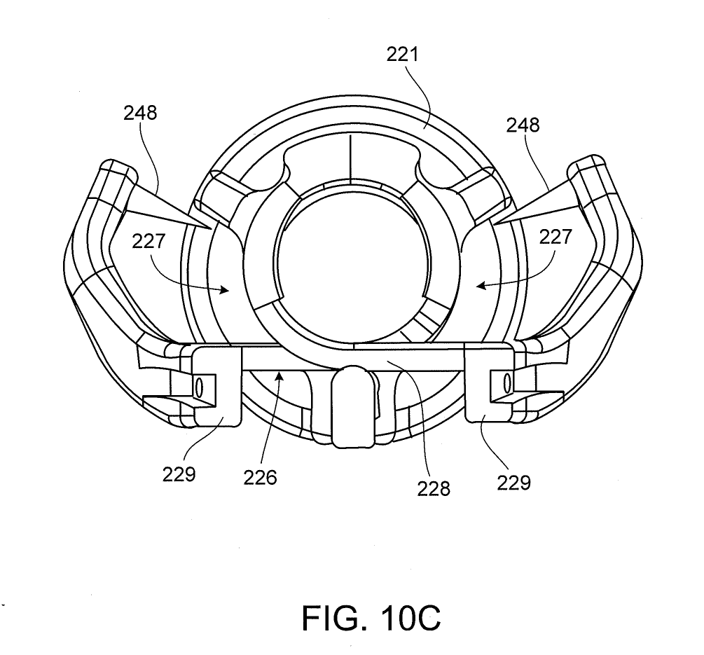

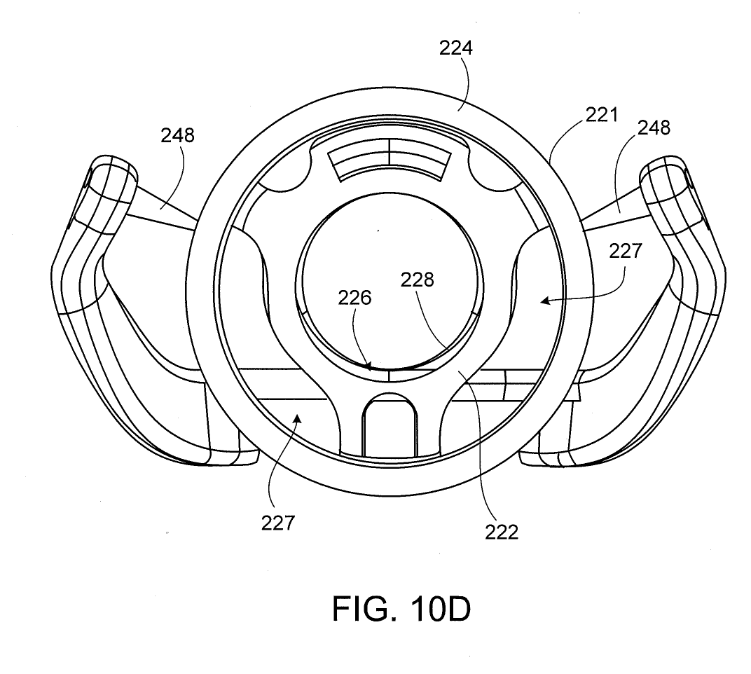

For example, FIGS. 10A-10D illustrate an implementation of a colpotomizer cup 220 that includes integrated anchors 248. The colpotomizer cup 220 also includes an annular body 221 (“cup body”), a cup base 222 at a proximal end, and a rim 224 at a distal end. The distal end 225 of the colpotomizer cup 220 is configured to receive a cervix, and the rim 224 is beveled to provide an anatomical landmark and incision backstop during use. A clamping mechanism 226 is connected to cup base 222. The anchors 248 are connected to the clamping mechanism 226 such that operation of the clamping mechanism 226 controls movements of the anchors 248. Viewing windows 227 are provided in the annular body 221. The clamping mechanism 226 includes a spring clamp 228 and tenaculum engagement members 229, which are disposed at opposite ends of the spring clamp 228. The engagement members 229 include recesses 230 for connecting the colpotomizer cup 220 to a tenaculum 240. Proximal ends 249 of the anchors 248 are attached to respective ones of the engagements members 229.

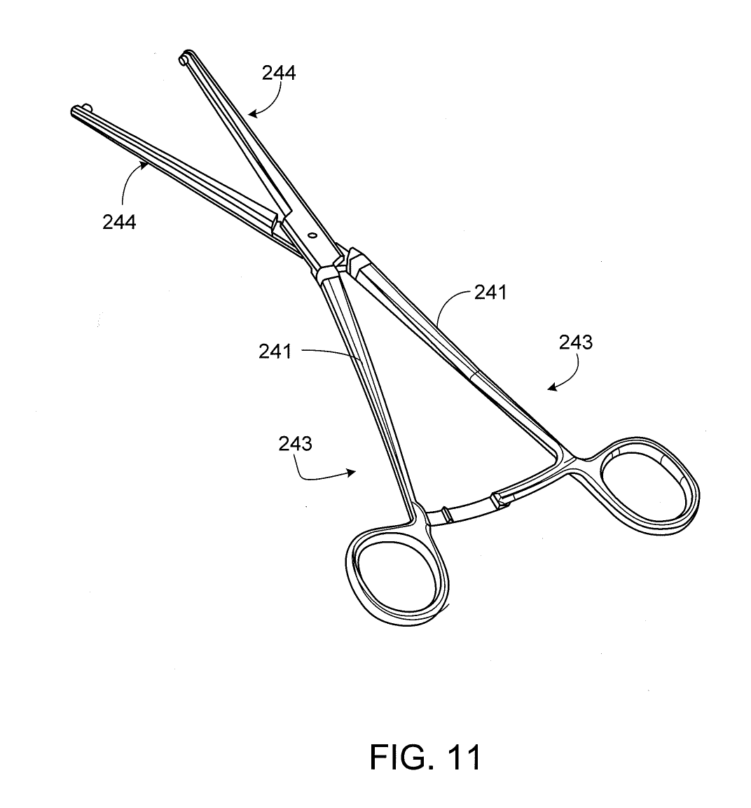

FIG. 11 illustrates a tenaculum 240 that can be used with the colpotomizer cup 220 of FIGS. 10A-10D. The tenaculum 240 includes a pair of legs 241 that are connected together. Distal ends 244 of the legs 241 are configured to engage the tenaculum engagement members 229 of the colpotomizer cup 220. When connected to the colpotomizer cup 220, the tenaculum 240 is operable to control operation of the clamping mechanism 226 as well as operation of the anchors 248. More specifically, with the colpotomizer cup 220 mounted to the tenaculum 240, the proximal leg portions 243 of the tenaculum 240 can be brought together to cause an opening 233 of the clamping mechanism 226 to enlarge (e.g., to receive the tip base 61 of manipulator tip 60 (FIG. 4)), as well as to cause the pointed distal ends of the anchors 248 to move inwardly toward each other, e.g., for piercing a cervix and thereby anchoring the colpotomizer cup 220 to the cervix.

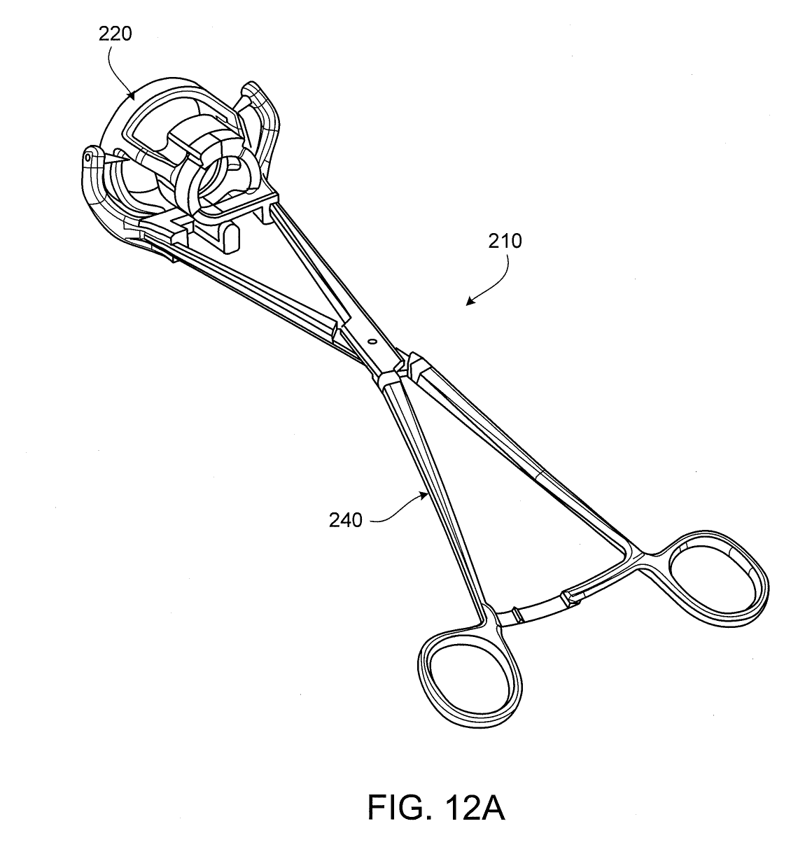

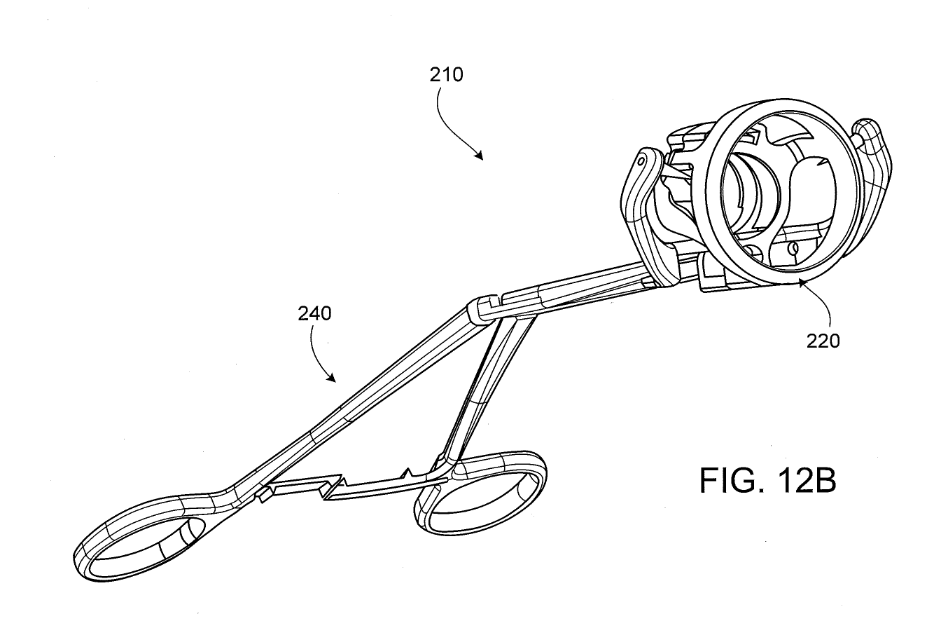

FIGS. 12A and 12B illustrate a colpotomizer cup delivery assembly 210 that includes the colpotomizer cup 220 and the tenaculum of 240.

While a method has been described in which the tenaculum is removed from the patient prior to incising the uterus. In some cases, the tenaculum may be left in the vagina during surgery.

While the use of the colpotomizer cup delivery assembly has been described in the context of hysterectomy surgery, the colpotomizer cup delivery assembly can be used for other types of surgeries or treatments.

Other implementations are within the scope of the following claims.

What is claimed is:

1. A colpotomizer cup comprising:
a cup body configured to receive a cervix; and
one or more tenaculum engagement features adapted to connect the colpotomizer cup to a tenaculum such that the colpotomizer cup can be delivered to a cervix using the tenaculum.

2. The colpotomizer cup of claim 1, wherein the one or more tenaculum engagement features are adapted to releasably connect the colpotomizer cup to a tenaculum such that the colpotomizer cup can be delivered to a cervix using the tenaculum.

3. The colpotomizer cup of claim 1, further comprising one or more anchors, said one or more anchors being operable to secure the colpotomizer cup to a cervix.

4. The colpotomizer cup of claim 1, wherein the colpotomizer cup includes a clamping mechanism, said clamping mechanism being operable to secure the colpotomizer cup to a manipulator tip of a uterine manipulator.

5. The colpotomizer cup of claim 4, wherein the clamping mechanism comprises the tenaculum engagement features.

6. The colpotomizer cup of claim 4, wherein the clamping mechanism comprises a spring clamp.

7. A colpotomizer cup delivery assembly, comprising:
a tenaculum; and
a colopotmizer cup connected to the tenaculum such that the colpotomizer cup can be delivered to a cervix using the tenaculum.

8. The colpotomizer cup delivery assembly of claim 7, wherein the colpotomizer cup includes one or more tenaculum engagement features adapted to releasably connect the colpotomizer cup to the tenaculum such that the colpotomizer cup can be delivered to a cervix using the tenaculum.

9. The colpotomizer cup delivery assembly of claim 7, wherein the colpotomizer cup includes one or more anchors, said one or more anchors being operable to secure the colpotomizer cup to a cervix.

10. The colpotomizer cup delivery assembly of claim 9, wherein the tenaculum is operable to control operation of the one or more anchors.

11. The colpotomizer cup delivery assembly of claim 7, wherein the tenaculum comprises anchors, said anchors being operable to secure the tenaculum and the connected colpotomizer cup to a cervix.

12. The colpotomizer cup delivery assembly of claim 7, wherein the colpotomizer cup includes a clamping mechanism, said clamping mechanism being operable to secure the colpotomizer cup to a manipulator tip of a uterine manipulator.

13. The colpotomizer cup delivery assembly of claim 12, wherein the clamping mechanism comprises one or more tenaculum engagement features for connecting the colpotomizer cup to the tenaculum.

14. The colpotomizer cup delivery assembly of claim 12, wherein the tenaculum is operable to control operation of the clamping mechanism.

15. The colpotomizer cup delivery assembly of claim 12, wherein the clamping mechanism comprises a spring clamp.

16. A method comprising:
inserting a colpotomizer cup into a vaginal cavity such that the colpotomizer cup receives a cervix; and then
inserting a manipulator tip of a uterine manipulator into the vaginal cavity such that a finger of the manipulator tip extends into an opening of the cervix.

17. The method of claim 16, further comprising clamping the colpotomizer cup to the manipulator tip.

18. The method of claim 16, further comprising maneuvering the tenaculum to clamp the colpotomizer cup to the manipulator tip.

19. The method of claim 16, further comprising maneuvering the tenaculum to operate a clamping mechanism and thereby clamping the colpotomizer cup to the manipulator tip.

20. The method claim 16, further comprising maneuvering the tenaculum to secure the colpotomizer cup to the cervix.

21. The method of claim 16, further comprising maneuvering the tenaculum to engage the cervix with one or more anchors.

22. The method of claim 16, further comprising removing the tenaculum from the vaginal cavity while the manipulator tip and the colpotomizer cup remain in contact with the cervix.

23. The method of claim 16, wherein inserting the colpotomizer cup into the vaginal cavity comprises inserting a tenaculum carrying the colpotomizer cup into the vaginal cavity and such that the colpotomizer cup receives the cervix.
